(19) United States
(12) Patent Application Publication
(10) Pub. No.: US 2022/0061883 A1
(43) Pub. Date: Mar. 3, 2022

(54) Locking Trocar Assembly

(71) Applicant: Sector 3, Hanover, PA (US)

(72) Inventors: Walter Seth Greene, Gettysburg, PA (US); Robert Clements Gillis, Gettysburg, PA (US)

(73) Assignee: Sector 3, Hanover, PA (US)

(21) Appl. No.: 17/411,453

(22) Filed: Aug. 25, 2021

Related U.S. Application Data
(60) Provisional application No. 63/069,994, filed on Aug. 25, 2020.

(51) Int. Cl. A61B 17/34; A61B 17/17

(52) U.S. Cl. CPC A61B 17/3421; A61B 2017/0046; A61B 2017/3456; A61B 17/17

(57) ABSTRACT

A locking trocar assembly having a cannulated drill guide with a proximal end and a distal end and a lumen extending therebetween, the cannulated drill guide having a handle near the proximal end, and a distal opening at the distal end; and an obturator having a proximal end and a distal end with an obturator shaft extending therebetween, the obturator shaft being configured to be directed through the lumen of the cannulated drill guide, the obturator having a cap at the proximal end of the obturator, the cap configured to be releasably secured to the handle of the cannula when the obturator shaft is directed through the lumen.

### CROSS-REFERENCE TO RELATED APPLICATIONS

This application claims the benefit of the filing date under 35 U.S.C. § 119 of the U.S. Provisional Patent Application No. 63/069,994, filed on Aug. 25, 2020.

### FIELD OF INVENTION

The field of the invention is directed to a locking trocar assembly, and a method of securing the components of a trocar in relative position, such that the trocar can be accurately advanced and precisely positioned within the body of a living being.

### BACKGROUND

The use of a percutaneous drill guide is commonplace in a variety of minimally invasive surgery procedures, for example shoulder surgery, such as labral repairs of the shoulder. There are many medical device companies who each have their own cannulated drill guide. The cannulated drill guides all work on the same principle of creating a guide that assists in the correct placement of a drill bit to be used during surgery. No matter the manufacturer, all percutaneous cannulated drill guides resemble a hollow screwdriver, having a hollow shaft through which a drill bit can be passed, and a handle by which the user can grasp, manipulate and control the drill guide.

When the drill guide is to be percutaneously inserted, it is initially placed on the skin, typically provided with an obturator passed through the cannulated drill guide in order to fill the interior lumen of the drill guide, and provide a dilating tip, and to approximate the qualities of a solid device. With an obturator inserted within the drill guide, both the obturator and the drill guide are able to advanced through the skin, as the tapered distal tip of the obturator serves to dilate an opening in the tissue, and continued advancement provides smoother passage of the drill guide through the skin and subcutaneous tissues. Without providing an obturator positioned within the lumen of the drill guide to dilate the tissue opening, it is very difficult to accurately pass the guide through the skin and subcutaneous tissue to deliver the drill guide in the appropriate position. Once the drill guide is placed correctly, the obturator may then be removed and a drill bit may then be passed down through the guide, for drilling a hole in the target location.

With the current devices on the market, the surgeon must manually hold the obturator in place as it and the drill guide are advanced through the skin and pass through underlying tissue. As the drill guide and obturator are advanced, the surgeon must maintain the obturator within the drill guide, using the surgeon's hand, thumbs and/or fingers to maintain the relative positions. If the surgeon fails to secure the obturator relative to the drill guide, there is nothing to prevent the obturator from being pushed out of the back of the drill guide as the components are advanced. The surgeon therefore must manually hold the drill guide handle and the obturator, which requires an awkward positioning of the surgeon's hand, in order to position or advance the drill guide, all while simultaneously preventing the obturator from moving backwards relative to the drill guide. This requirement can cause a significant control issue for the tool as it must be held in a certain position to prevent the trocar from ‘backing out’ while attempting to pass the guide through the skin and subcutaneous tissues to the proper position.

What is needed is a device that can securely maintain the position of the obturator while inserted into the drill guide, thereby freeing the surgeon from having to grip the device in a manner that simultaneously secures the obturator within the drill guide, while advancing the drill guide into the precise position for use.

### SUMMARY

A novel locking trocar assembly is provided and is used for securing the relative positions of the trocar components while the trocar is advanced and positioned within the body of a living being.

In an exemplary embodiment, there is provided a locking trocar assembly having a cannulated drill guide with a proximal end and a distal end and a lumen extending therebetween, the cannulated drill guide having a handle near the proximal end, and a distal opening at the distal end; and an obturator having a proximal end and a distal end with an obturator shaft extending therebetween, the obturator shaft being configured to be directed through the lumen of the cannulated drill guide, the obturator having a cap at the proximal end of the obturator, the cap configured to be releasably secured to the handle of the cannula when the obturator shaft is directed through the lumen.

### BRIEF DESCRIPTION OF THE DRAWINGS

The invention will now be described by way of example with reference to the accompanying Figures of which:

FIG. 1 is a perspective view of a cannula according to an exemplary embodiment of the invention;

FIG. 2 is a cross-sectional view of the cannula of FIG. 1;

FIG. 3 is a perspective view of an obturator having a sharp tip, according to an exemplary embodiment of the invention;

FIG. 4 is a perspective view of an obturator having a blunt tip, according to an exemplary embodiment of the invention;

FIG. 5 is a perspective view of the locking trocar assembly according to an exemplary embodiment of the invention;

FIG. 6 is a cross-sectional view of the locking trocar assembly of FIG. 5;

FIG. 7 is a perspective view of another locking trocar assembly according to an exemplary embodiment of the invention;

FIG. 8 is a cross-sectional view of the locking trocar assembly of FIG. 7;

FIG. 9 is a perspective view of a locking trocar assembly according to another exemplary embodiment of the invention;

FIG. 10 is a top perspective view of the isolated cannula of the locking trocar assembly of FIG. 9;

FIG. 11 is a partial perspective view of the disassembled components of the locking trocar assembly of FIG. 9;

FIG. 12 is a cross-sectional view of the locking trocar assembly taken along line 12-12 of FIG. 9;

FIG. 13 is another cross-sectional view of the locking trocar assembly of FIG. 12, only with the obturator having been rotated slightly to releasably lock the obturator in place within the cannula;

FIG. 14 is a cross-section view of the locking trocar assembly taken along line 14-14 of FIG. 12; and

FIG. 15 is a cross-section view of the locking trocar assembly taken along line 15-15 of FIG. 13.

### DETAILED DESCRIPTION OF THE EMBODIMENTS

Exemplary embodiments of the invention will be described hereinafter in detail with reference to the attached drawings, wherein like reference numerals refer to like elements.

The invention may, however, be embodied in many different forms and should not be construed as being limited to the particular embodiments set forth herein; rather, these embodiments are provided so that the present disclosure will be thorough and complete, and will fully convey the concept of the disclosure to those skilled in the art.

The invention is for structural improvements that are in relation to the percutaneous trocar assemblies. The trocar assembly provides a cannulated drill guide, and an obturator that can be removably received within the interior of the cannulated drill guide. The invention can be utilized with trocar assemblies utilizing any suitable obturator, including those having a sharpened distal tip, as well as those having a rounded, or blunted distal tip. The invention provides for the reversible securement of the obturator within the cannulated drill guide, utilizing the embodiments described below, to secure the cap of the obturator to the handle of the cannulated drill guide. With the obturator secured to the cannulated drill guide, the distal portion of the trocar assembly can be percutaneously inserted, without the need for the user to actively maintain the relative positions of the trocar components, as the securement ensures that the obturator will remain in place while the cannulated drill guide is advanced into the tissue, and preventing the obturator from backing out of the cannulated drill guide as the distal tip of the obturator encounters resistance from the tissue being penetrated. The securement of the obturator to the cannulated drill guide is configured to be releasable, such that the obturator cap can be disengaged from the handle of the drill guide, whereupon the obturator can be withdrawn from the drill guide once placed in position within the body of a being. Thus in an embodiment of the invention, the trocar assembly provides for the temporary unification of the obturator and cannulated drill guide, using any suitable locking mechanism to secure the two components together and ensure that the obturator and the cannulated drill guide will perform as a single unit. In an embodiment, as will be discussed below, the securement of the obturator is achieved by providing on the obturator a threaded screw portion that can engage with a complementary threaded screw portion on the handle of the cannula that allows the obturator to ‘lock-in-place’ within the cannulated drill guide. In another embodiment, the obturator and the cannulated drill guide are locked together with a non-threaded fastener, as will be discussed below. The structural improvements will allow the components of the locking trocar assemblies to be reversibly secured, thereby effectively obviating the need for the obturator to be manually held in place by the surgeon's grip while the cannulated drill guide is being inserted into a being.

In this manner, the various embodiments described herein address the issues with requiring the surgeons to control both components of a trocar device while being passing through the skin and subcutaneous tissues. This novel design has the obturator reversibly attached to the cannulated drill guide, thereby freeing the surgeon's hand to allow the placement of the trocar assembly at any angle while still maintaining the purpose of the obturator of allowing the cannulated drill guide to perform as a solid unit. Prior to the invention described herein, a trocar assembly of a cannulated drill guide and an obturator would have been required to have the surgeon maintain the obturator in position using his grip. The addition of a locking mechanism between the obturator and the cannulated drill guide will allow the surgeon to have more control while passing the trocar assembly through tissue, freeing the surgeon's grip from securing the components together, which will lead to shorter operating room times and the facilitation of overall improvements to patient care and safety. Various embodiments of the invention are further shown with reference to the attached drawings.

### First Exemplary Embodiment

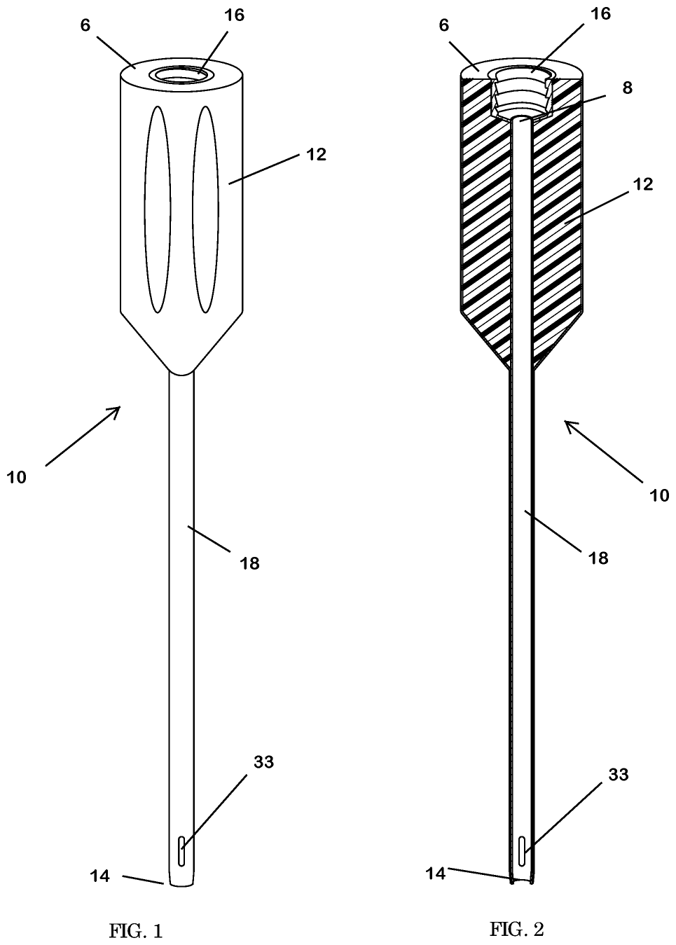

FIGS. 1-8 establish the components and overall structure of a locking trocar assembly, according to a first exemplary embodiment of the invention.

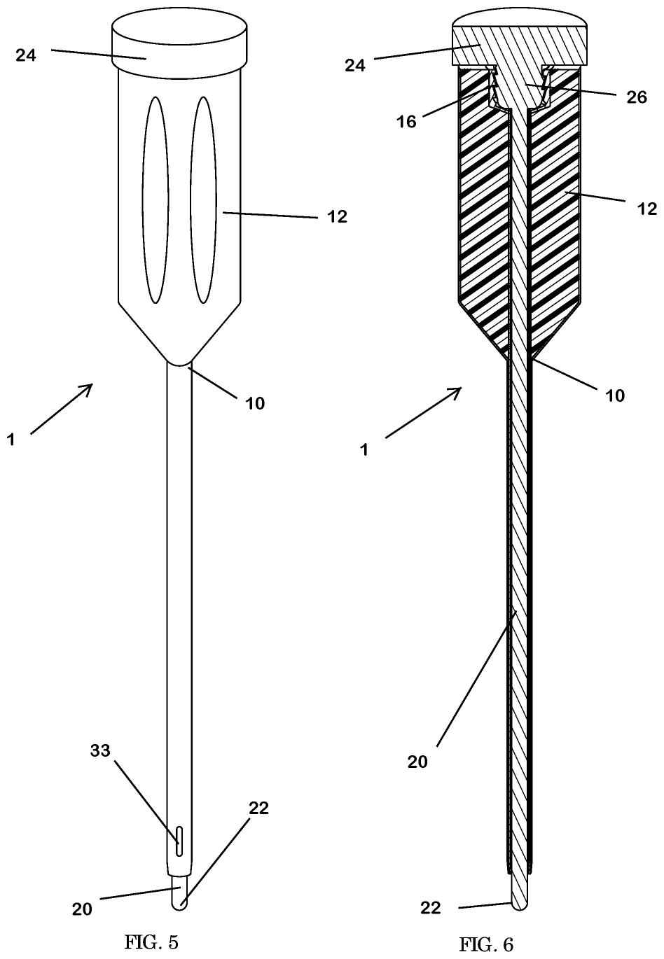

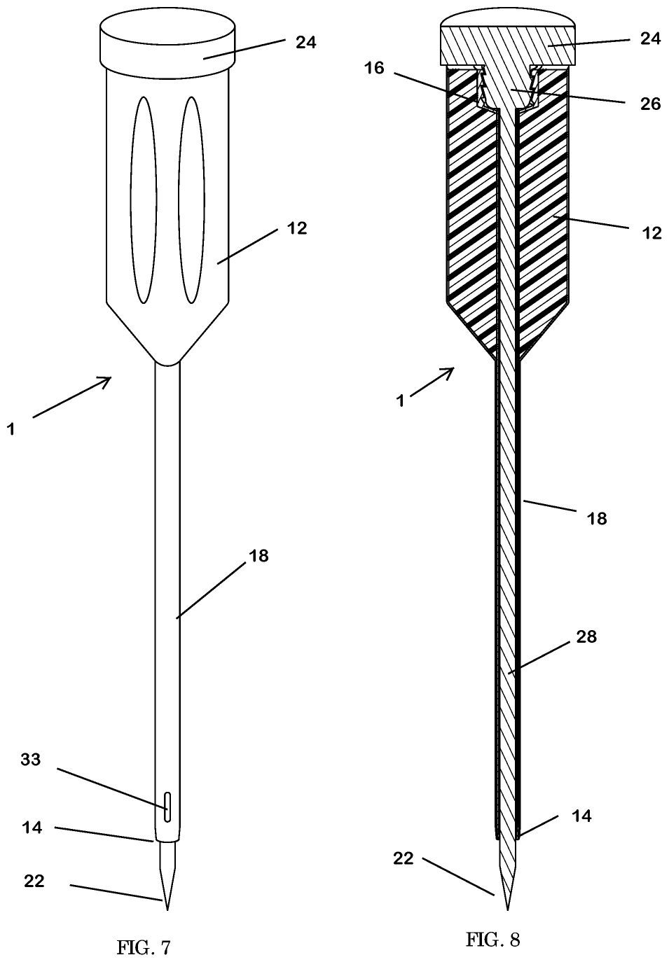

In the shown embodiment, and with reference to FIGS. 5-8, the locking trocar assembly 1 is shown having a cannulated drill guide 10, provided with an obturator 20 inserted through a cannula shaft 18. As depicted, the obturator 20 is reversibly secured to a handle 12 of the cannulated drill guide 10, as will be discussed. Specifically, an embodiment of an assembled trocar assembly 1 having the obturator 20 provided with a blunt distal tip 22 is depicted in FIG. 5, and in cross-sectional view in FIG. 6. FIG. 7 shows a similar cannulated drill guide 10, however the obturator 20 is provided having a distal tip 22 that is sharp or bladed. FIG. 8 shows a cross-section view of the locking trocar assembly 1 of FIG. 7. The design or features of the distal tip 22 in any embodiment, may instead by any variety of alternative obturators as are well known to those of skill in the art, and may be utilized similarly according to the teachings here.

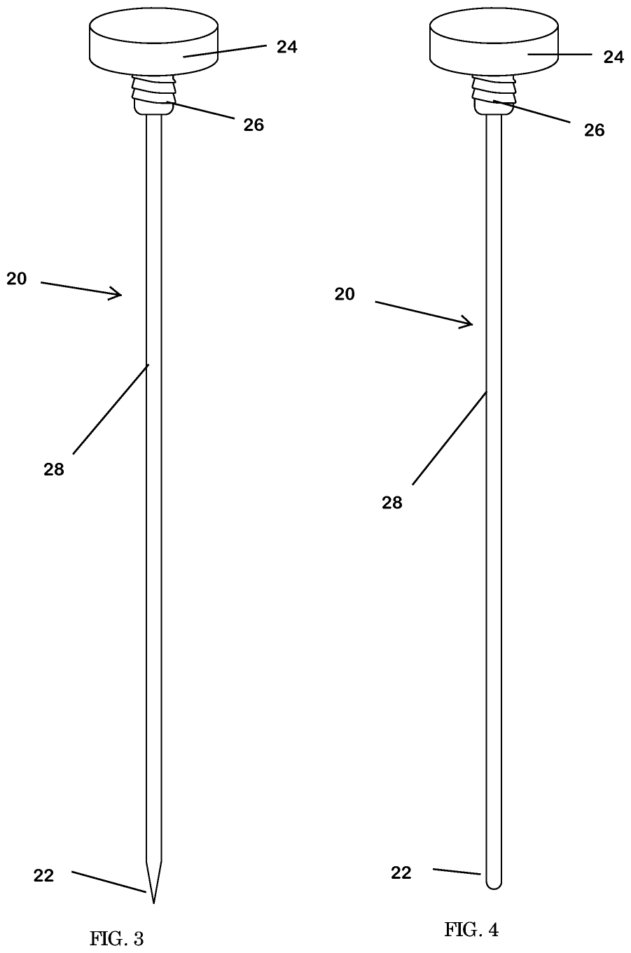

With reference to FIG. 4, there is shown an obturator 20 in isolation, where the obturator has a distal tip 22 that has a rounded, or blunt end. Similarly, with reference to FIG. 3, there is shown an obturator 20, now having a distal tip 22 that is a sharp or bladed (e.g., pyramidal) tip. The obturator 20 depicted in each of FIGS. 3 and 4, is an awl-like device, configured to puncture into soft tissue and optionally bone, and is shown having an obturator shaft 28, a cap 24, and a protruding portion 26. The protruding portion 26 is at least partly threaded, shown here as having an externally threaded (e.g., male threaded) portion. The shaft 28 of the obturator 20 is secured to the cap 24 in any suitable manner, for example, embedded within the protruding portion 26 and/or cap 24, as shown in FIGS. 3 and 4. The outer surface of the cap 24 may be provided with knurling, or grooves, or any suitable grip elements to improve the operator's grip upon the cap, and provide better tactile feel.

With reference to FIG. 1, a cannulated drill guide 10 is depicted in isolation, and includes a cannula shaft 18 and a handle 12. FIG. 2 shows a cross-section view of the cannulated drill guide 10 of FIG. 1. The cannula shaft 18 may be any suitable construction for a cannula, as will be familiar to those of skill in the art. The cannula shaft 18 as shown, is a hollow tube, having an interior lumen. In an embodiment, the cannula shaft 18 is a substantially straight-sided lumen, though as depicted in FIGS. 1 and 2, the lumen may optionally be tapered inwards slightly leading to a narrowed distal opening 14 relative to the balance of the shaft 18, such that distal opening may seal against the obturator as it is passed through the distal opening 14. It is also contemplated that the shaft 18 may be straight-sided, and not required to taper towards the distal opening (similar to the end of the shaft 18 shown in FIG. 12) Optionally, the cannula shaft 18 may be provided one or more fenestration ports 33 through the sidewall of the catheter shaft 18. As shown in FIG. 1, the fenestration port 33 is at a position that is proximal from, but near the distal opening 14 of the cannula shaft 18. The cannula shaft 18 has a proximal opening 8 and is secured at its proximal end to the handle 12. The handle is any suitable form and construction, as will be familiar to those of skill in the art, and may be, for example, generally cylindrical, and aligned along the longitudinal axis of the cannula. The handle may be provided with knurling, or grooves, or any suitable grip elements that will provide additional tactile control over the trocar assembly 1 as it is being manipulated by the user. The cannula shaft 18 is secured to the handle 12 in any suitable manner, for example, embedded within the handle 12, as shown in FIG. 2. At the proximal face 6 of the handle 12, there is provided a receiving cavity 16, for receiving therein the protruding portion 26 of the obturator 20. The receiving cavity 16 is provided with a threaded portion that is to be complementary to the threaded portion of the protruding portion 26, such that the cap 24 and handle 12 can be threaded together, as will be discussed. As depicted in FIG. 2, the receiving cavity 16 features internal threads (e.g., female threads) over at least a portion of the receiving cavity 16 sidewall that can engage with the protruding portion 26 of the obturator 20.

The distal tip 22 of the obturator 20 is to pass through the receiving cavity 16 and be inserted into proximal opening 8 of the cannula shaft 18. The obturator may then be advanced further into the cannulated drill guide 10 until the protruding portion 26 encounters the receiving cavity 16 of the handle 12. The engagement features of the locking trocar assembly may then be engaged to secure the obturator 20 in position within the cannulated drill guide 10. For example, as shown with reference to FIG. 6, the cap 24 may be rotated such that the complementary threads of the protruding portion 26 are caused to engage with the threads of the receiving cavity 16. Continued rotation of the cap (relative to the handle 12) will cause the threads of the protruding portion 26 to advance into the receiving cavity, until the cap 24 rests against the proximal face 6 of the handle 12. The combined length of the shaft 28 and protruding portion 26 of the obturator 20 is longer than the length of the cannulated drill guide 10, in order to ensure that at least the distal tip 22 of the obturator 20 protrudes from the distal opening 14 of the cannula shaft 18 when the obturator 20 is seated within the cannulated drill guide 10, and the locking trocar assembly is fully assembled.

With the threads of the obturator engaged with the complementary threads of the handle, the trocar assembly may be advanced, and the obturator is prevented from backing out of the cannulated drill guide by the engaged threads. Thus, the surgeon need not maintain a grip that simultaneously manipulates the trocar assembly, and secures the components together, rather the surgeon need only concentrate on the positioning of the trocar assembly.

Removal of the obturator 20 from the cannula drill guide can easily be achieved by reversing the rotation to disengage the threads of the handle 12 and the protruding portion 26 of the obturator 20. In this manner, surgeon may maintain the handle in one position, and with the other hand, reverse the rotation of the cap 24 to disengage the connector elements to allow the obturator to be removed.

### Second Exemplary Embodiment

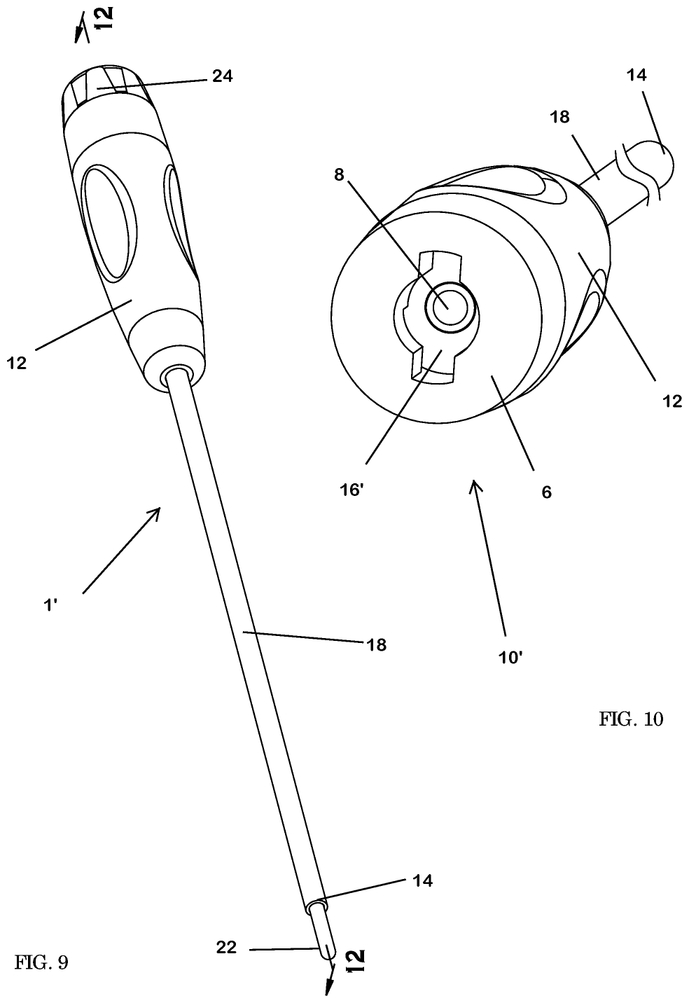

In another exemplary embodiment according to the invention, and with reference to FIGS. 9-15, a locking trocar assembly 1′ is provided and includes a non-threaded securement mechanism, as will be discussed, for reversibly securing together the obturator 20′ and cannulated drill guide 10′.

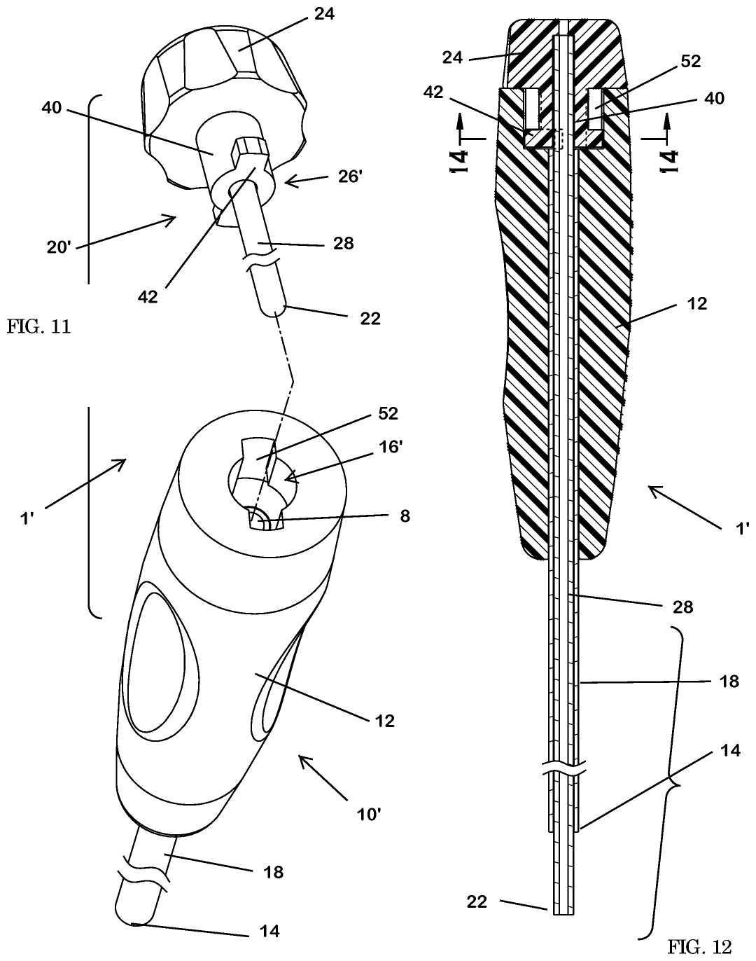

With reference to FIG. 11, the locking trocar assembly 1′ also features an obturator 20′, that is configured to be inserted into, and can be releasably secured relative to a cannulated drill guide 10′. The cannulated drill guide 10′ and the obturator 20′ are substantially similar to the above described components, however, with the following differences. Rather than provide a threaded engagement of the obturator 20 with the cannulated drill guide 10, as has been previously described, the embodiment of the locking trocar assembly 1′ relies on a non-threaded securement mechanism for reversibly joining together the obturator 20′ and cannulated drill guide 10′. As shown, the cap 24, mounted at the proximal end of the obturator shaft 28 has a protruding portion 26′ that can be received within a complementary receiving cavity 16′ of the handle 12 of the cannulated drill guide 10′. It is contemplated that the alternate embodiment of the locking trocar assembly 1′, thus can be seen to provide a keyed connection, such as a bayonet mount or a twist lock connection. As shown with reference to FIG. 11, the protruding portion 26′, provides a male connector element having a barrel 40, with at least one radially protruding element 42 (e.g. pin or bayonet). As depicted, the cylindrical barrel 40 has a pair of opposing radially protruding elements 42, arranged on opposite sides of the barrel 40, though it is contemplated that more protruding elements 42 may optionally be provided. With a portion of the obturator shaft 28, including the distal tip 22 inserted into the interior lumen of the cannula shaft 18, the cap 24 is approximated to the handle 12. The cap 24, as it is caused to be rotated about the longitudinal axis in line with the obturator shaft 28, will bring the radially protruding element(s) 42 into alignment with a corresponding opening or set of openings in the proximal face of the handle 12, depicted here as slot 52 of the receiving cavity 16′. In this manner, there is provided a keyed connection for the protruding portion 26′ and receiving cavity 16′, as the protruding elements 42 and slot 52 must be indexed to a point that they are in general alignment to allow the insertion of the male connector of the obturator 20′ into the female connector of the cannulated drill guide 10′. The slot 52, in an embodiment, is located at the proximal face 6, or near the proximal face 6 of the handle 12, as depicted in FIGS. 10 and 11, and receiving cavity 16′ extends distally into the handle 12, below the slot 52. Once the male and female connectors are in proper alignment, the connectors are advanced together, bringing the cap 24 near to, or into contact with the proximal face 6 of the handle 12, as shown in FIGS. 9 and 12, with the distal tip 22 of the obturator extending out the distal opening of the cannula shaft 18. As can be seen with reference to FIG. 12, and in cross-section view of the handle in FIG. 14, the protruding element(s) 42 is to pass through the slot 52, and into a position below the slot 52, as depicted in FIG. 12, yet remain in vertical alignment with the slot 52. So long as the cap 24 has not been rotated, and thus remains rotationally aligned in the insertion position, the engagement of the connectors can easily be reversed by pulling upwards on the cap 24, relative to the handle 12, to cause the protruding elements 42 to be retracted back through the slot 52, and initiate the retraction of the obturator 20′ at least partially out of the cannula drill guide 10′.

As can be seen with reference to FIG. 14, the receiving cavity 16′ within the handle 12, and at a position below (or distal to) the slot 52, provides locking passages 54 within the handle 12. The locking passages 54 are not in vertical alignment with the slot 52, and are obscured by the proximal face 6 in the view shown in FIG. 10.

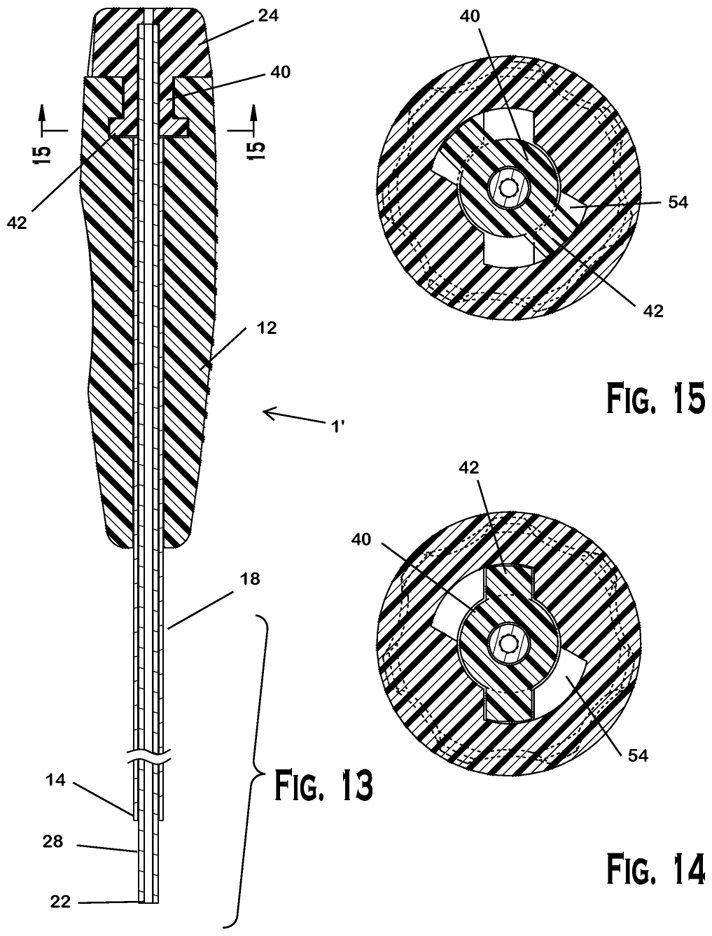

With reference to FIGS. 13 and 15, the cap 24, where the protruding elements 42 have been passed through the slot 52 in a distal direction (e.g., with the cap in contact with the proximal face 6), the cap 24 may then be caused to be rotated, whereupon the protruding elements 42 will be rotated and caused to enter into the locking passages 54, as shown. The rotation of the cap, and of the protruding elements 42, as depicted in FIGS. 13 and 15, is of an amount that brings the protruding element(s) 42 out of vertical alignment with the slot 52, and at least partially into the locking passages 54. While the cap 24 is rotated away from the insertion alignment, the protruding elements 42 cannot be retracted out of the slot 52, as they remain out of alignment with each other. Thus, the cap and the handle are secured together, such that as the trocar assembly is advanced, the protruding elements 42 will serve to prevent backing out of the obturator 20′ from the cannula drill guide 10′.

Rotation of the cap to the original insertion alignment will cause the protruding elements 42 to be brought back into the position that is in vertical alignment with the slot 52, as shown in FIGS. 12 and 14, such that the removal of the obturator can be achieved by pulling up on the cap, relative to the handle 12.

In use, the distal tip of the obturator is advanced into the cannula's proximal end, and advanced along the length of the cannula until the cap is positioned as shown in FIG. 9, and the distal tip of the obturator is extended out the distal opening of the cannula. The cap may then be partially rotated about the longitudinal axis of the obturator, relative to the cannula handle, to be secured within the receiving cavity of the handle, as will be discussed. However, once the user desires to disengage the connector, the male connector is rotated into a position that the protruding element(s) is returned to alignment with the slot of the receiver, whereupon the components may be disengaged by passing the protruding element(s) through the slot as the male connector is retracted out of the receiver.

In an embodiment, the cap is provided with a plurality of protruding elements, such as two or more pins that can be aligned with a corresponding number of slots in the receiving portion of the handle. It is further contemplated that the connector may have a plurality of slots that can allow the connection of the cap to the handle, e.g. where the pins are placed at angles 90 and 270, it is contemplated that the slots may be provided to accommodate the connection with slots at 0 and 180, as well as a second set of slots oriented at 90 and 270, thus the user may rotate the cap through a maximum of a quarter turn in order to bring the connection into suitable alignment. One skilled in the art will recognize that variations to the connection described herein are possible, yet fall within the spirit of the invention.

The foregoing illustrates some of the possibilities for practicing the invention. Many other embodiments and fields of use for the locking trocar assembly 1 are possible and within the scope and spirit of the invention. It is, therefore, intended that the foregoing description be regarded as illustrative rather than limiting.

What is claimed is:

1. A trocar assembly comprising:
a cannula having a proximal end and a distal end and a lumen extending therebetween, the cannula having a handle near the proximal end, and a distal opening at the distal end,
and an obturator having a proximal end and a distal end with an obturator shaft extending therebetween, the obturator shaft being configured to be directed through the lumen of the cannula, the obturator having a cap at the proximal end of the obturator, the cap configured to be releasably secured to the handle of the cannula when the obturator shaft is directed through the lumen.

2. The trocar assembly of claim 1, wherein the cannula is a substantially rigid, straight-sided tube.

3. The trocar assembly of claim 2, wherein the cannula is a drill guide.

4. The trocar assembly of claim 1, wherein the handle has a receiving cavity at the proximal end of the cannula.

5. The trocar assembly of claim 4, wherein the cap has a protruding portion to releasably engage with the receiving cavity, such that when the protruding portion is engaged with the receiving cavity, the obturator is secured within the cannula.

6. The trocar assembly of claim 5, wherein the cap and the handle are secured relative to each other by a fastening means selected from the group consisting of threaded elements, keyed components.

7. The trocar assembly of claim 6, wherein the protruding portion of the cap is a male fastener means, and the receiving cavity is a female fastening means.

8. The trocar assembly of claim 7, wherein the male fastener means comprises an externally threaded surface.

9. The trocar assembly of claim 8, wherein the female fastening means comprises an internally threaded surface.

10. The trocar assembly of claim 7, wherein the male fastening means comprises male bayonet connector.

11. The trocar assembly of claim 10, wherein the female fastening means comprises a female bayonet connector.

12. The trocar assembly of claim 1, wherein the cannula is embedded in the handle.

13. The trocar assembly of claim 1, wherein the obturator has a distal tip that is sharp.

14. The trocar assembly of claim 1, wherein the obturator has a distal tip that is blunt.

15. The trocar assembly of claim 1, wherein at least one of the cannula and obturator are configured to be disposable.

16. The trocar assembly of claim 1, wherein the cannula has at least one fenestration.
